(19) United States
(12) Patent Application Publication
(10) Pub. No.: US 2003/0139710 A1
(43) Pub. Date: Jul. 24, 2003

(54) Disposal feminine personal protection panties

(76) Inventor: Lynda G. Patterson, Chicago, IL (US)

Correspondence Address: Lynda G. Patterson, 8214 S. St. Lawrence, Chicago, IL, 60619-5010, US

(21) Appl. No.: 10/053,828

(22) Filed: Jan. 24, 2002

(51) Int. Cl. A61F 13/15

(52) U.S. Cl. 604/367; 604/389

(57) ABSTRACT

A disposal feminine protection garment with a panty area (10) attached directly to an absorbable protection pad (12). The feminine protection panties is constructed of materials that look and feel like a regular garment. Adhesive strips can be added for ease in removal and placement (14). Disposal feminine personal protection panties provide superior protection from the staining of other clothing and linens. There is greater comfort and convenience with a single one-wear disposal garment.

### CROSS-REFERENCE TO RELATED APPLICATIONS

Not applicable

### BACKGROUND

1. Field of the Invention

This invention relates to making available a more convenient means of providing feminine personal protection.

2. Description of Prior Art

Modern day feminine protection products started as heavy pads that were maintained in position with uncomfortable belts. In many instances pads move, resulting in spills and the destruction of fine lingerie, clothing, and linen.

The next generation of feminine protection products is pads of various sizes and thickness with an adhesive strip, a great improvement, but still these pads are subject to a high failure rate. Failure can result because of placement error and/or adhesive failure. Sometimes these products have a tendency to bunch or move resulting in spillage. To relieve the incidental staining of clothing, wings were added to feminine personal protection products. At times poor product design can cause accidental soilage of clothing, lingerie or linen. One example is wing design. Wings can be too long or wings can be too short on the feminine protection pad.

Now feminine personal protection comes in light, moderate, heavy and overnight. It is provided with or without wings. Nevertheless all of the above feminine personal protection products hereto are known to suffer from a number of disadvantages.

The Main Problems Are:

a) The present feminine protection even when adhered to a regular panty can move because of the movement of the panty itself causing spillage and soilage of clothing, lingerie and/or linen.

b) The wings sometimes fail. Wings can be too long or wings can be too short on the feminine protection pad. When wings fail spillage can be the consequence.

c) The present feminine protection feels like an extra garment even the lightest ones.

d) The present feminine protection products are bulky.

### SUMMARY

The disposal feminine personal panties is a one-wear disposal garment providing feminine protection and eliminating the soiling of other clothing, lingerie and linen, which will provide added convenience. This product can be constructed of paper, natural fibers, synthetics or any combination of the above materials. It is designed to look and feel like panties. The panty can be folded like today's contemporary pads to be easily transportable.

Objects and Advantages According, several objects and advantages of the disposable feminine personal protection panties are:

a) The feminine protection panties are more convenient, like the progression from women's hosiery to panty hose. In the past, garter belts were used to hold up a woman's stocking, the next generation of women's hosiery was the panty hose. The logical progression of feminine protection is disposal panties styled with the protection pad as an actual part of the garment. The one-wear disposal panties eliminate the need for belts, adhesives and wings.

b) The availability of a product that offers superior protection and eliminates damage to other garments and linen. The feminine protection panties have been designed with (FIGS. 3,4,5 6 and 7) and without (FIGS. 1 and 2 ) adhesive side tabs. The panties without the side tabs are especially designed for light days, overnights and travel. The panty with the tabs is designed for ease in changing during the day or during times of heavy flow.

c) To provide added comfort because it is an all in one product. the extra comfort is that it is one garment. A garment that is one wear and thrown away.

d) Feminine panties can be manufactured folded so that they are easy to transport (FIGS. 8A, B, C, and D).

e) This product can be universally used as a travel garment one-wear and thrown away. So often we travel and have to wash out under-garments. With this invention it can be worn once and thrown away. The protection panties can be manufactured in all absorbencies from panty liners to overnight.

### DRAWING FIGURES

FIG. 1 shows a front view of the feminine personal protection panties. The feminine protection pad as actually a part of the garment. Number 10 is the panty area and number 12 is the protection area.

FIG. 2 demonstrates a top view of the feminine personal protection panties. With the feminine protection pad as actually a part of the garment. Number 10 is the panty area and number 12 is the absorbable protection area.

FIG. 3 illustrates a front view of the feminine personal protection panties. With the feminine protection pad as actually a part of the garment and adhesive tabs. Number 10 is the panty area and number 12 is the absorbable protection area. Number 14 is the adhesive tab.

FIG. 4 illustrates a top view of the feminine personal protection panties. The feminine protection pad as actually a part of the garment and adhesive tabs are demonstrated. Number 10 is the panty area and number 12 is the absorbable protection area. Number 14 is the adhesive tab.

FIG. 5 shows the feminine personal protection panties open from the outside view. The feminine protection pad as actually a part of the garment and adhesive tabs are demonstrated. Number 10 is the panty area and number 14 is the adhesive tab.

FIG. 6 shows the feminine personal protection panties open from the inside view. The feminine protection pad as actually a part of the garment and adhesive tabs are demonstrated. Number 10 is the panty area and number 12 is the absorbable protection area. Number 14 is the adhesive tab.

FIG. 7 shows the feminine personal protection panties from the side view. The feminine protection pad as actually a part of the garment and adhesive tabs are demonstrated. Number 10 is the panty area and number 12 is the absorbable protection area. Number 14 is the adhesive tab.

FIGS. 8A, B. C, and D demonstrates the folding of feminine protection panties.

### REFERENCE NUMBER IN DRAWING

10 panty area

12 absorbable protection area

14 adhesive tabs

### DESCRIPTION

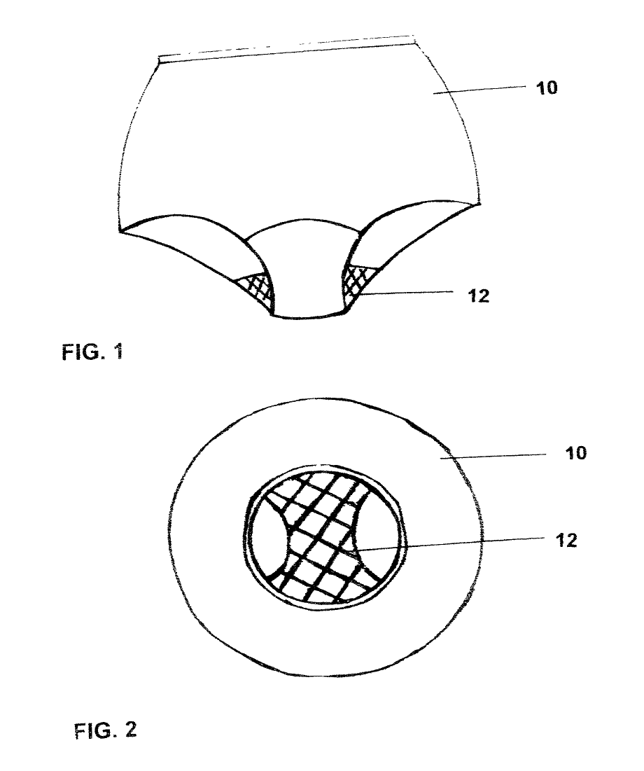

FIGS. 1 and 2—Primary Design

The present invention as illustrated in FIG. 1 (front view) and FIG. 2 (top view) shows the basis components of the feminine protection panties. The panty area can be composed of paper, natural fibers, synthetics and/or any combination of the above. The styles of the panties are briefs, bikini, high-cut, thong, and etc 10.

Directly attached to a panties is a protective pad, which is provided in all absorbencies from panty liners to overnight and obstetric 12.

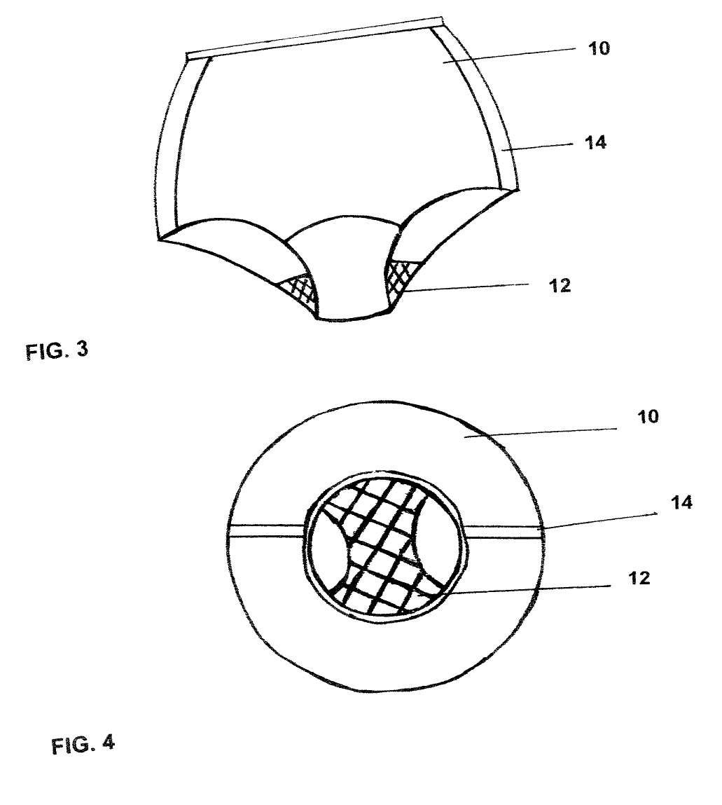

FIGS. 3, 4, 5, 6, and 7—Secondary Design

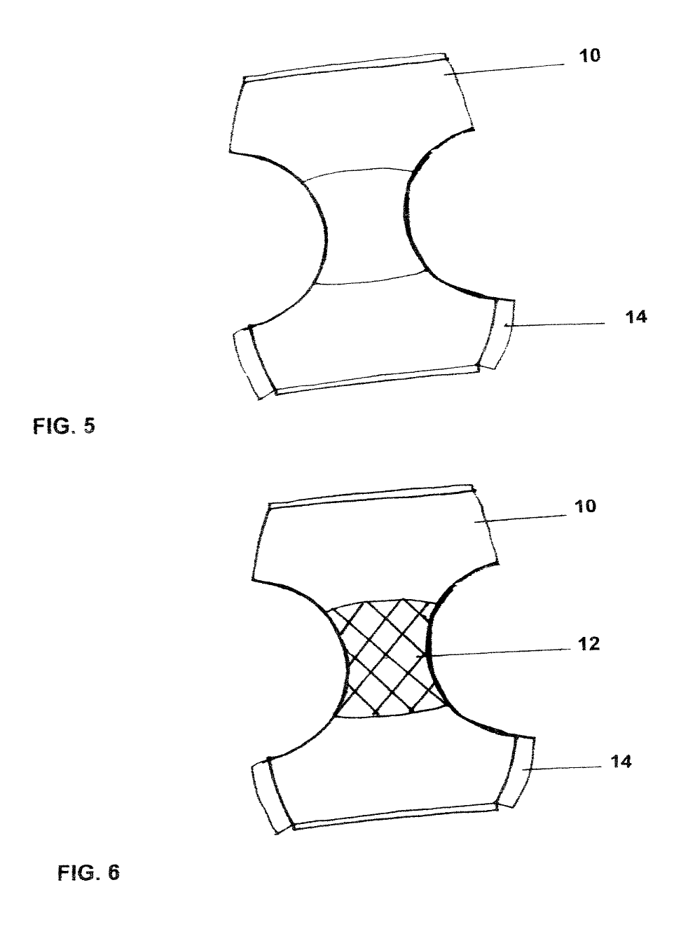

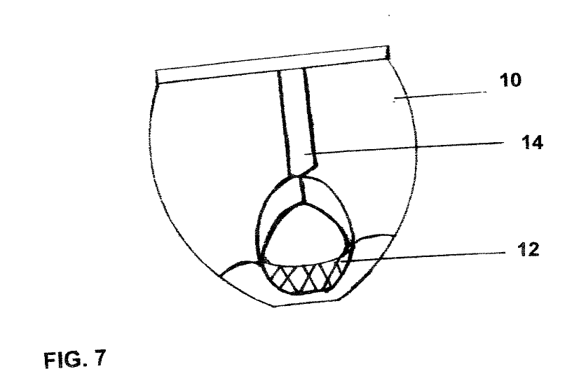

The ease in removal and placement of the feminine protection panties can be achieved with an adhesive tab(s) 14 FIG. 3 (front view) FIG. 4 (top view) FIG. 5 (outside view) FIG. 6 (inside view) and FIG. 7 (side view).

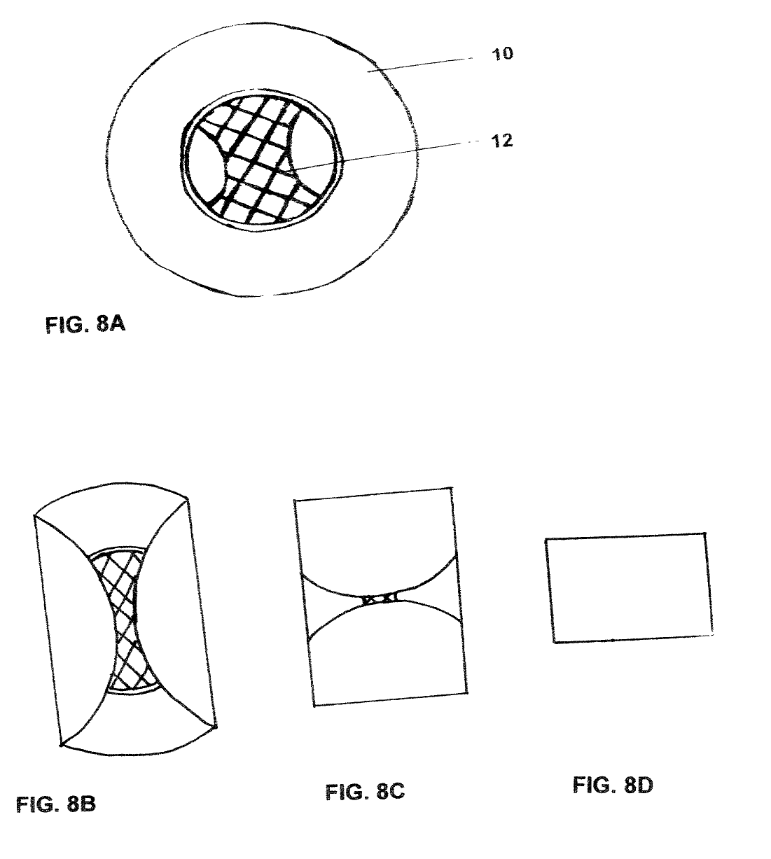

FIGS. 8A, B. C, D

The present invention as demonstrated shows ease in folding FIGS. 8A, B, C, and D for ease in transporting (manufactured folded).

Conclusion

Accordingly, the reader will see that the feminine disposal protection panties can be used easily and conveniently.

it provides superior protection from spillage.

it provides superior protection from soilage of other garments and bed linens.

it provides superior comfort by being a single garment that is worn once and disposed of.

it provides convenience as a disposal travel garment.

it looks and feels like panties.

the primary design is especially intended for travel, light days and overnights, but can be worn at other times.

the secondary design is for ease in remove especially for daytimes and active times, but can be worn at other times.

I claim:

1. The feminine protection panties are an all in one-wear disposal product that offers greater protection and comfort.

2. The feminine protection panties is designed to look and feel like a panties. It can be constructed of paper, natural fibers, synthetics or any combination of the above products. The feminine protection without the adhesive tab can be used especially for light days and overnights, but can be worn at other times.

3. The feminine protection panties with adhesive tabs can provide ease in changing especially for daytimes and active times, but can be worn at other times.

4. The feminine protection panties provide superior protection by eliminating the need to wear other garments thereby no damage can occur.

5. The feminine protection panties can be manufactured folded for ease in transportability.

6. The feminine protection panties can be worn universally by all women, the can be worn as travel panties or as panties in general.
